(19) United States
(12) Patent Application Publication
(10) Pub. No.: US 2024/0316219 A1
(43) Pub. Date: Sep. 26, 2024

(54) COMPOSITIONS AND METHODS RELATED TO THE TREATMENT OF OCULAR DISEASES IN EQUINES

(71) Applicants: NORTH CAROLINA STATE UNIVERSITY, Raleigh, NC (US); THE UNIVERSITY OF NORTH CAROLINA AT CHAPEL HILL, Chapel Hill, NC (US)

(72) Inventors: Brian Gilger, Raleigh, NC (US); Elizabeth Crabtree, Raleigh, NC (US); Matthew L. Hirsch, Chapel Hill, NC (US)

(21) Appl. No.: 18/558,919

(22) PCT Filed: May 2, 2022
(86) PCT No.: PCT/US22/27283

§ 371 Date: Nov. 3, 2023

Related U.S. Application Data
(60) Provisional application No. 63/183,234, filed on May 3, 2021.

(51) Int. Cl. A61K 48/00; A61K 9/00; A61P 27/02; C07K 14/54; C12N 15/86

(52) U.S. Cl. CPC A61K 48/005; A61K 9/0048; A61K 48/0075; A61P 27/02; C07K 14/5428; C12N 15/86; C12N 2750/14143

(57) ABSTRACT

The present disclosure provides compositions and methods related to the treatment of ocular diseases in equines. In particular, the present disclosure provides novel compositions and methods related to the administration of therapeutic compositions comprising AAV-equine IL-10 for the treatment and/or prevention of various ocular diseases (e.g., non-infectious uveitis).

### CROSS REFERENCE TO RELATED APPLICATIONS

This application claims priority to and the benefit of U.S. Provisional Patent Application No. 63/183,234 filed May 3, 2021, which is incorporated herein by reference in its entirety for all purposes.

### GOVERNMENT SUPPORT

This invention was made with government support under grant number KR211915 awarded by the National Institutes of Health. The government has certain rights in the invention.

### FIELD

The present disclosure provides compositions and methods related to the treatment of ocular diseases in equines. In particular, the present disclosure provides novel compositions and methods related to the administration of therapeutic compositions comprising equine IL-10 for the treatment and/or prevention of various ocular diseases (e.g., non-infectious uveitis).

### BACKGROUND

Equine recurrent uveitis (ERU) is a spontaneous, non-infectious, painful and sight-threatening disease affecting up to 25% of equine populations worldwide. This disease is characterized by episodes of active ocular inflammation alternating with varying intervals of clinical quiescence. Research in horses, humans, and laboratory animals has shown that non-infectious recurrent uveitis have a T-cell mediated inflammatory response with a multifactorial origin, related to the environmental factors and genetic makeup of an individual. Recurrent uveitis in horses develops following primary uveitis when disruption to the blood-ocular barrier occurs, allowing CD4+T-lymphocytes to enter and remain in the eye. This disruption enables host immune responses to react to ocular self-antigens that are not normally recognized by the immune system, and subsequent episodes of uveitis develop as a consequence of new antigenic detection. The accumulated effects of recurrent “bouts” or “flares” of inflammation leads to progressively destructive pathologic changes including irreversible scarring, ocular cloudiness, cataract formation, and vision loss. Conventional treatment of ERU is non-specific, including frequent use of topical and systemic corticosteroids and other immunosuppressive agents; none of which are effective in preventing uveitis relapses. These therapies are limited by poor treatment compliance and long-term adverse effects, such as corneal degeneration, glaucoma, cataract, ocular hypertension, and infection, all of which may contribute to development of blindness.

Experimental autoimmune uveitis (EAU) animal rodent models have been valuable in the study of non-infectious, immune mediated uveitis. EAU is mediated predominantly by CD4+T-lymphocytes, and proinflammatory cytokines, which is very similar to the inflammatory profile in naturally occurring uveitis in humans and horses. These models have proven useful in developing therapeutics to treat or prevent the immune inflammatory responses within the eye. Blocking these proinflammatory T-cells and enhancing anti-inflammatory T-cell cytokine function within the eye, could halt the inflammatory cytokine cascade and therefore would be an effective target for treating immune mediated uveitis.

### SUMMARY

Embodiments of the present disclosure include a composition for treating ocular disease and other non-infectious inflammatory diseases in equines. In accordance with these embodiments, the composition includes an adeno-associated virus (AAV) vector comprising a polynucleotide encoding an equine IL-10 polypeptide, or a functional derivative or variant thereof, and a pharmaceutically acceptable carrier and/or excipient. In some embodiments, the composition is suitable for ocular administration to an equid.

In some embodiments, the polynucleotide encoding the equine IL-10 polypeptide is codon optimized. In some embodiments, the polynucleotide encoding the equine IL-10 polypeptide is at least 75% identical to SEQ ID NO: 1. In some embodiments, the polynucleotide encoding the equine IL-10 polypeptide comprises SEQ ID NO: 1.

In some embodiments, the IL-10 polypeptide has at least 85% identity with SEQ ID NO: 2. In some embodiments, the IL-10 polypeptide comprises SEQ ID NO: 2.

In some embodiments, the AAV vector is at least one of an AAV serotype 1 (AAV1) vector, an AAV serotype 2 (AAV2) vector, an AAV serotype 3B (AAV3B) vector, an AAV serotype 4 (AAV4) vector, an AAV serotype 5 (AAV5) vector, an AAV serotype 6 (AAV6) vector, an AAV serotype 7 (AAV7) vector, an AAV serotype 8 (AAV8) vector, an AAV serotype 9 (AAV9) vector, or a derivative or variant thereof.

In some embodiments, the composition is administered by injection into a portion of the subject's eye or by direct application (e.g., topical application) of the composition to a portion of the subject's eye.

In some embodiments, the composition is administered intravitreally (IVT), intracorneally, subconjunctivally, periocularly, suprachoroidally, intrasclerally, intracamerally, intravenously, and/or subretinally. In some embodiments, the composition is administered at a dose of at least 1.0×10⁹ vg.

In some embodiments, the composition further comprises a buffer. In some embodiments, the composition further comprises a surfactant. In some embodiments, the composition comprises a pH from about 4.0 to about 8.0.

In some embodiments, the composition further comprises a biologically active agent. In some embodiments, the biologically active agent is selected from the group consisting of an immunosuppressant, an NSAID, a steroid, an antibacterial, and any combination thereof. In some embodiments, the steroid is dexamethasone or prednisone, and any combination thereof. In some embodiments, the NSAID is selected from the group consisting of flunixin meglumine, phenylbutazone, firocoxib, diclofenac, flurbiprofen, bromfenac, nepafenac, and any combination thereof. In some embodiments, the immunosuppressant is selected from the group consisting of cyclosporin, tacrolimus (FK506), rapamycin (sirolimus), infliximab, bevacizumab, and any combination thereof. In some embodiments, the antibiotic is selected from the group consisting of gentamicin, tobramycin, amikacin, ceftazidime, vancomycin, and any combination thereof.

In some embodiments, the AAV vector further comprises a polynucleotide encoding an immunomodulating agent selected from the group consisting of: TGFβ, an IL-1 receptor antagonist, IL-33, IL-35, IL-37, IDO-1, and any combination thereof.

Embodiments of the present disclosure also include a kit comprising any of the compositions described herein, and at least one container. In some embodiments, the at least one container comprises a syringe and a needle suitable for administration to an equine. In some embodiments, the kit further comprises instructions for administration to an equine.

Embodiments of the present disclosure also include a method of treating or preventing an ocular or inflammatory disease in equines. In accordance with these embodiments, the method includes administering any of the compositions described herein to an equine.

In some embodiments, the ocular disease causes blindness, impaired vision, and/or ocular pain, and administration of the composition treats and/or prevents the blindness, impaired vision, and/or ocular pain. In some embodiments, the treating and/or the preventing of blindness comprises lymphocyte suppression.

In some embodiments, the ocular disease comprises uveitis, immune-mediated keratitis, heterochromic iridocyclitis with keratitis, endothelitis, posterior uveitis, chorioretinitis, optic neuritis, and any combination thereof. In some embodiments, the uveitis is recurrent, chronic, non-infectious uveitis.

In some embodiments of the method, the composition is administered by injection into a portion of the subject's eye or by direct application of the composition to a portion of the subject's eye. In some embodiments of the method, the composition is administered intravitreally (IVT), intracorneally, subconjunctivally, periocularly, suprachoroidally, intrasclerally, intracamerally, intravenously, and/or subretinally. In some embodiments of the method, the composition is administered at a dose of at least 1.0×10⁹ vg. In some embodiments of the method, the composition is administered in a single dose, and wherein the single dose treats and/or prevents at least one symptom associated with the ocular or inflammatory disease. In some embodiments of the method, the at least one symptom comprises ocular cloudiness, blindness, impaired vision, and/or ocular pain.

### BRIEF DESCRIPTION OF THE DRAWINGS

FIG. 1: AAV8-Equine IL-10 Improves EAU clinical scores. Rats were treated by intravitreal (IVT) injections with scAAV8-Equine-IL-10 low dose or high dose (n=5 rats, 10 eyes) or treated with BSS (n=4 rats, 8 eyes). One week after IVT injections, EAU was induced by immunization with IRBP and ocular inflammation was examined by slit lamp biomicroscopy. Bar graph of EAU mean clinical exam score for each rat revealed that EAU clinical scores peaked on days 12, 13, and 14 after uveitis induction. Mean clinical scores were significantly lower on days 12-14 in the Equine-IL-10 high dose (2.4×10¹⁰ vg) treated rats compared to the BSS rats and clinical scores were significantly lower on days 10-14 in the Equine-IL-10 low dose (2.4×10⁹ vg) treated rats compared to BSS rats. (p=0.002 to 0.049); Pairwise Wilcoxon tests). There were no significant differences in mean clinical scores between high dose or low dose rats treated with scAAV8-Equine-IL-10 on any day.

FIG. 2: AAV8-Equine-IL-10 Improves EAU inflammatory cell count in the anterior chamber. Optical coherence tomography (OCT) was performed once prior intravitreal injections, once prior induction of EAU and then every other day following induction of EAU. Scatter plot of EAU inflammatory cell count of each rat revealed that there was evidence of cellular infiltrate in the anterior chamber of rats on days 10, 12 and 14 following induction of EAU. Mean inflammatory cell count were significantly less in rats treated with a single intravitreal injection of scAAV8-Equine-IL-10 (high or low dose) compared to BSS rats on days 10, 12 and 14 post EAU induction (p=<0.004 to 0.043); Pairwise Wilcoxon tests). There were no significant differences in mean inflammatory cell count between high or low dose treated rats on any day.

FIGS. 3A-3E: AAV8-Equine-IL-10 Improves EAU histology scores. Representative images of ocular histology demonstrate iris thickening and inflammatory cell infiltration in the ciliary body, iris, anterior chamber and vitreous body, as well as moderate vasculitis formation in experimental autoimmune uveitis (EAU) BSS eyes (A). Mild to no infiltration of inflammatory cells was observed in the iris or ciliary body of high dose and low dose Equine-IL-10 treated EAU eyes (B and C respectively: hematoxylin & eosin staining; original magnification: 10×). The histological infiltrative scores (D) and structural scores (E) were significantly decreased in both low and high dose scAAV8-Equine-IL-10 treated eyes (n=5 eyes) as compared to the BSS-EAU eyes (n=4 eyes) (p=0.010, p=0.015; Wilcoxon test). Each point is the average score of an individual eye of two blinded observers and mean scores of each treatment group are denoted by the horizontal bars.

FIGS. 4A-4B: AAV8-Equine-IL-10 expression and ocular distribution. (A) Equine-IL-10 abundance examination by qRT-PCR in selected tissues are presented as vector cDNA/host transcript (GAPDH) (One-way ANOVA; * p<0.01). Results represent experiments done in triplicate with mean value expressed. (B) Vector genome copy number in distinct ocular tissues are shown as vector genome copy number/ug of host genome DNA (One-way ANOVA; * p<0.0l).

FIG. 5: Representative images of retinal histopathology. Representative images of ocular histology demonstrate inflammatory cell infiltration vitreous body, and retina in experimental autoimmune uveitis (EAU) BSS eyes, with almost infiltration of inflammatory cells observed high dose and low dose Equine-IL-10 treated EAU eyes (hematoxylin & eosin staining; original magnification: 20×).

FIG. 6: Representative OCT images of each group of rat on day 12 post EAU induction. The red circles used to demonstrate cells in the anterior chamber. The iris, cornea and anterior chamber (AC) are labeled in the top right image.

FIG. 7: Equine-IL-10 Western Blot. A Western blot was used to detect Equine-IL-10 protein following transfection of human embryonic kidney 293 cells (HEK293). Equine-IL-10 protein (eq-IL-10) was detected in the supernatant of cultured HEK293 cells (KDa (kilodaltons); GFP (green fluorescent protein); p459 (Equine IL-10 plasmid)).

FIGS. 8A-8B: Representative data from equine IL-10 ELISA assays. Interpolated data from equine-IL-10 dilution 1:16 had a higher standard deviation and had data points higher than the standard curve; therefore, data from the 1:32 dilution was used to determine the concentration of the Equine-IL-10 supernatant (A). Average Interpolated data from Equine-IL-10, 1:32 dilution=9.7 ng/mL concentration. Final supernatant concentration of Equine-IL-10 (interpolated concentration multiplied by dilution factor: 32) was approximately 310.4 ng/mL (B).

FIG. 9: Representative data demonstrating equine IL-10 suppression of T-lymphocytes. T-lymphocytes were extracted from equine plasma and incubated for 4 days in at ˜37° C. with 3 different concentrations of Equine-IL-10 supernatant (100 ng/mL, 50 ng/mL and 1 ng/mL). Controls: T-lymphocytes+ConA w/o equine-IL-10 (positive control); T-lymphocytes+HEK cell supernatant w/o Equine-IL-10.

FIG. 10: Representative data from expression studies performed in various ocular tissues in the rat EAU model. Rats were administered AAV8-eqIL-10 by intravitreal injection at a low (1.2×10⁹ vg) or a high (1.2×10¹⁰ vg) dose (FIG. 10).

### DETAILED DESCRIPTION

Embodiments of present disclosure provide compositions and methods related to the treatment of ocular diseases in equines. In particular, the present disclosure provides novel compositions and methods related to the administration of therapeutic compositions comprising equine IL-10 for the treatment and/or prevention of various ocular diseases (e.g., non-infectious uveitis). Equine recurrent uveitis (ERU) is a chronic, intractable, ocular disease that is considered to be one of the most common causes of blindness in horses. Current treatments of ERU are non-specific and have many side effects which limits them to short-term use. In order to develop an effective therapy for ERU, experiments were conducted to investigate the use of adeno-associated virus (AAV) gene therapy, exploiting a natural immune tolerance mechanism induced by equine interleukin-10 (Equine-IL-10).

As described further herein, both low and high doses of AAV-Equine-IL-10 administered to the eyes demonstrated a significant decrease in clinical inflammatory scores and AH cell counts compared to saline-treated EAU eyes on days 10, 12 and 14 post EAU induction. Mean cellular histologic infiltrative scores were also significantly less in AAV-Equine-IL-10 dosed eyes compared to saline control eyes. Intravitreal injection of AAV8-Equine-IL-10 resulted in Equine-IL-10 cDNA expression the ciliary body and retina of both treatment groups. A dose dependent influence of cDNA expression in the cornea and optic nerve was observed. Taken together, these results demonstrate that a single IVT injection of AAV8-Equine-IL-10 inhibited EAU in the Lewis rat. These results supports further evaluation of this innovative therapy for ERU and other types of non-infectious uveitis.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having.” “has,” “can,” “contain(s),” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” with respect to the compositions described herein generally refers to providing a composition of the present disclosure to a subject in need of treatment. The compositions of the present disclosure are generally administered to a subject's eye (ocular administration). In some embodiments, the compositions can be administered by injection into a portion of a subject's eye or by direct application of the composition to a portion of the subject's eye. For example, the composition can be administered (e.g., via injection) intravitreally (IVT), intracorneally, subconjunctivally, periocularly, suprachoroidally, intrasclerally, intracamerally, intravenously, and/or subretinally. In other embodiments, the composition is formulated as a medicament that is applied directly to a portion of a subject's eye (e.g., topical application). Routes of systemic administration are also possible, in accordance with the compositions and methods described herein.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions (e.g., suitable for IVT injection). Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A. or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

As used herein, the term “subject” and “patient” as used herein interchangeably refers to any vertebrate, including, but not limited to, cow, pig, camel, llama, horse, donkey, mule, zebra, goat, rabbit, sheep, hamsters, guinea pig, cat, dog, rat, mouse, non-human primates, and humans. In some embodiments, the subject may be an equid, which refers to any species from the genus Equus, including but not limited to, horses donkeys, zebras, and mules, and any variant thereof. The subject or patient may be undergoing various forms of treatment separate and independent of the methods described herein.

As used herein, the term “treat,” “treating” or “treatment” are each used interchangeably herein to describe reversing, alleviating, or inhibiting the progress of a disease and/or injury, or one or more symptoms of such disease, to which such term applies, and/or to improve/enhance one or more aspects of a subject's physical health Depending on the condition of the subject, the term also refers to preventing a disease, and includes preventing the onset of a disease, or preventing the symptoms associated with a disease (e.g., ocular disease). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, the term “salts” and “pharmaceutically acceptable salts” generally refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disuSfonic, oxalic, and isethionic, and the like. Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, isopropanol, and the like. Lists of suitable salts can be found, for example, in Remington's Pharmaceutical Sciences, 17th ed., Mack Publishing Company, Easton, Pa., 985.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Kits

The results of the present disclosure demonstrate that intravitreal injections of AAV8-Equine-IL-10 suppressed the development of induced uveitis as determined by reduced clinical inflammatory scores, reduced OCT inflammatory cell counts, and reduced histopathological scores in an experimental autoimmune uveitis Lewis rat model. ERU is the leading cause of progressive blindness in horses with few effective and safe long term preventative or treatment options. Recurrent bouts of intraocular inflammation lead to progressive damage within the eye, which in turn, leads to significant economic loss and utility for ERU horse owners; as a result affected horses are often euthanized. Results provided herein demonstrate the efficacy of ocular gene therapy using AAV as a more effective treatment strategy for recurrent immune mediated ocular inflammation. These data support that gene therapy would reduce or eliminate the need for topical corticosteroids and systemic anti-inflammatories, thus mitigating the risks of side effects and systemic immune suppression, while still reducing ocular inflammation and preserving long term vision.

As demonstrated further herein, the eye has unique advantages for the use of gene therapy: it is readily accessible and has a blood ocular barrier that limits both an immune response and systemic redistribution of intraocular therapeutics. Study results demonstrated that both a high and lose dose intravitreal delivery of Equine-IL-10 gene resulted in expression of Equine-IL-10 cDNA in the ciliary body/iris and retina, which corresponded with decreased clinical and histological inflammatory scores. Interestingly, the high dose group of rats also exhibited viral expression in the cornea and optic nerve, whereas the low dose groups did not. This indicated a dose dependent influence for viral distribution of ocular tissues via intravitreal injection. Previous studies have demonstrated that several factors influence AAV's affinity for specific tissue transduction including different AAV serotype and route of injection. AAV8 has established affinity for both the cornea and optic nerve following subconjunctival or intracameral injections. It is important to note that the target tissue, the iris/ciliary body, was efficiently transduced by both the low and high dose groups. The ciliary body was targeted because it is the location of the blood ocular barrier and localized IL-10 at the blood ocular barrier could aid in stabilizing the barrier and maintain the eye's immune privilege state.

IL-10 incites immune tolerance by directly inhibiting macrophages, natural killer, Th1 and dendritic cell function. Dysregulation of IL-10 is associated with an increased response to infection but also an increased risk for development of many autoimmune diseases. Several studies have evaluated the anti-inflammatory and immunosuppressive effects of IL-10 both as a systemic and local treatment. Systemic administration of IL-10 has been evaluated as a treatment for patients with immune-mediated inflammatory diseases such as psoriasis, Crohn's disease (CD), and rheumatoid arthritis with trends toward efficacy in both psoriasis and CD. In horses, intra-articular injection of AAV5-Equine-IL-10 was effective in modulating synovial inflammation, with no systemic or localized adverse effects.

In regard to ocular therapy, experimental autoimmune uveitis (EAU), a uveitis rodent disease model, is a predominant T-cell immune-mediated disease, similar to horses with ERU. In vivo studies have demonstrated that IL-10 mRNA coincides with a decreased T-cell response. A report in 2005 demonstrated that gene therapy using AAV expressing IL-10 was successful in reducing EAU disease severity in an EAU model following a single subretinal injection. Another study in 2008 demonstrated that intracameral injection of Lentiviral-vector mediated expression of IL-10 reduced inflammatory cell infiltrate and protein content in a mouse uveitis model suggesting that localized IL-10 aids in maintaining integrity of the blood ocular barrier. Other studies have also demonstrated that subconjunctival and systemic administration of IL-10 have beneficial effects on uveitis models. These studies support that IL-10 has an important role in the downregulation of ocular inflammation and in the maintenance of ocular immune privilege.

In agreement with these studies, the data provided herein demonstrate that AAV8-Equine-IL-10 treated EAU rats experienced a significant reduction in ocular inflammation as judged by reduced clinical scores, decreased AH cellular infiltration on OCT, and decreased histological examination scores relative to control rats. Though both groups effectively reduced clinical score and inflammatory cell counts within the eye, the low dose group significantly reduced clinical scores compared to control rats two days before the high dose group. There was no significant difference between the low dose or high dose groups' clinical scores on any days. Inherently, a goal for clinical application is to determine the lowest possible effective therapeutic dose. The results of the present disclosure demonstrate that the lower dose group (1.2×10⁹ vector genomes [vg]) was effective in inhibiting intraocular inflammation and transducing the targeted tissue with no observed intraocular or systemic side effects. Based on what is currently known in the art, the results provided herein indicate that this is the first report to demonstrate the use of AAV-Equine-IL-10 as an equine specific DNA used as a single intravitreal injection for treatment of uveitis. Because Equine-IL-10 is a cytokine already naturally present in the equine eye, no long-term adverse effects of this optimized Equine-IL-10 are expected in the horse.

Thus, the compositions and methods of the present disclosure demonstrate that intravitreal delivery of a single dose of scAAV8-Equine-IL-10 established protection against ocular inflammation in EAU Lewis rats. In accordance with these embodiments, the compositions of the present disclosure include an adeno-associated virus (AAV) vector comprising a polynucleotide encoding an equine IL-10 polypeptide, or a functional derivative or variant thereof, and a pharmaceutically acceptable carrier and/or excipient. In some embodiments, the composition is suitable for ocular administration to an equid.

In some embodiments, the polynucleotide encoding the equine IL-10 polypeptide is codon optimized. In some embodiments, the polynucleotide encoding the equine IL-10 polypeptide is at least 75% identical to SEQ ID NO: 1. In some embodiments, the polynucleotide encoding the equine IL-10 polypeptide is at least 80% identical to SEQ ID NO: 1. In some embodiments, the polynucleotide encoding the equine IL-10 polypeptide is at least 85% identical to SEQ ID NO: 1. In some embodiments, the polynucleotide encoding the equine IL-10 polypeptide is at least 90% identical to SEQ ID NO: 1. In some embodiments, the polynucleotide encoding the equine IL-10 polypeptide is at least 91% identical to SEQ ID NO: 1. In some embodiments, the polynucleotide encoding the equine IL-10 polypeptide is at least 92% identical to SEQ ID NO: 1. In some embodiments, the polynucleotide encoding the equine IL-10 polypeptide is at least 93% identical to SEQ ID NO: 1. In some embodiments, the polynucleotide encoding the equine IL-10 polypeptide is at least 94% identical to SEQ ID NO: 1. In some embodiments, the polynucleotide encoding the equine IL-10 polypeptide is at least 95% identical to SEQ ID NO: 1. In some embodiments, the polynucleotide encoding the equine IL-10 polypeptide is at least 96% identical to SEQ ID NO: 1. In some embodiments, the polynucleotide encoding the equine IL-10 polypeptide is at least 97% identical to SEQ ID NO: 1. In some embodiments, the polynucleotide encoding the equine IL-10 polypeptide is at least 98% identical to SEQ ID NO: 1. In some embodiments, the polynucleotide encoding the equine IL-10 polypeptide is at least 99% identical to SEQ ID NO: 1. In some embodiments, the polynucleotide encoding the equine IL-10 polypeptide comprises SEQ ID NO: 1.

In some embodiments, the IL-10 polypeptide encoded by one or more of the IL-10 polynucleotides described above has at least 85% identity with SEQ ID NO: 2. In some embodiments, the IL-10 polypeptide has at least 90% identity with SEQ ID NO: 2. In some embodiments, the IL-10 polypeptide has at least 91% identity with SEQ ID NO: 2. In some embodiments, the IL-10 polypeptide has at least 92% identity with SEQ ID NO: 2. In some embodiments, the IL-10 polypeptide has at least 93% identity with SEQ ID NO: 2. In some embodiments, the IL-10 polypeptide has at least 94% identity with SEQ ID NO: 2. In some embodiments, the IL-10 polypeptide has at least 95% identity with SEQ ID NO: 2. In some embodiments, the IL-10 polypeptide has at least 96% identity with SEQ ID NO: 2. In some embodiments, the IL-10 polypeptide comprises SEQ ID NO: 2. In some embodiments, the IL-10 polypeptide has at least 97% identity with SEQ ID NO: 2. In some embodiments, the IL-10 polypeptide has at least 98% identity with SEQ ID NO: 2. In some embodiments, the IL-10 polypeptide has at least 99% identity with SEQ ID NO: 2. In some embodiments, the IL-10 polypeptide comprises SEQ ID NO: 2.

In some embodiments, the IL-10 polynucleotides of the present disclosure are administered to an equine using a gene delivery vector, such as, but not limited to, an Adeno-associated virus (AAV) vector. Adeno-associated virus is a member of the Parvoviridae family and comprises a linear, single-stranded DNA genome of less than about 5,000 nucleotides. AAV requires co-infection with a helper virus (i.e. an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired.

In some embodiments, the AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep-52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter.

The nucleic acid sequences encoding the equine IL-10 polypeptides of the present disclosure can be generated using methods known in the art. For example, nucleic acid sequences, polypeptides, and proteins can be recombinantly produced using standard recombinant DNA methodology (see, e.g., Sambrook et al., Molecular Cloning: A Laboratory Manual, 3^{rd} ed., Cold Spring Harbor Press, Cold Spring Harbor, N.Y., 2001; and Ausubel et al., Current Protocols in Molecular Biology, Greene Publishing Associates and John Wiley & Sons, N Y, 1994). Further, a synthetically produced nucleic acid sequence encoding an equine IL-10 can be isolated and/or purified from a source, such as a bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified.

In addition to the nucleic acid sequences encoding the equine IL-10 polypeptides of the present disclosure, the AAV vector generally comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego. Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e. initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ System (Stratagene, San Diego, Calif.), and the Cre-ERT tamoxifen inducible recombinase system (Indra et al., Nuc. Acid. Res., 27: 4324-4327 (1999); Nuc. Acid Res., 28: e99 (2000); U.S. Pat. No. 7,112,715; and Kramer & Fussenegger, Methods Mol. Biol., 308: 123-144 (2005)).

The term “enhancer” as used herein, generally refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In some embodiments, the nucleic acid sequence encoding the equine IL-10 is operably linked to a CMV enhancer/chicken β-actin promoter (also referred to as a “CAG promoter”) (see, e.g., Niwa et al., Gene. 108: 193-199 (1991); Daly et al., Proc. Natl. Acad. Sci. U.S.A., 96: 2296-2300 (1999); and Sondhi et al., Mol. Ther., 15: 481491 (2007)).

In accordance with the above, AAV vectors of the present disclosure can include, but are not limited to, at least one of an AAV serotype 1 (AAV1) vector, an AAV serotype 2 (AAV2) vector, an AAV serotype 3B (AAV3B) vector, an AAV serotype 4 (AAV4) vector, an AAV serotype 5 (AAV5) vector, an AAV serotype 6 (AAV6) vector, an AAV serotype 7 (AAV7) vector, an AAV serotype 8 (AAV8) vector, an AAV serotype 9 (AAV9) vector, or a derivative or variant thereof. Any of these AAV vectors can be used to deliver the equine IL-10 polynucleotides of the present disclosure. In some embodiments, AAV8 is used to deliver the equine IL-10 polynucleotides of the present disclosure, as provided further herein.

Additionally, embodiments of the present disclosure include delivering compositions comprising equine IL-10 polynucleotides by any means that are suitable to treat an ocular disease or condition. For example, equine IL-10 polynucleotides can be administered by injection of the composition into a portion of the subject's eye, and/or by direct application of the composition to a portion of the subject's eye. In some embodiments, the composition can be administered intravitreally (IVT), intracorneally, subconjunctivally, periocularly, suprachoroidally, intraselerally, intracamerally, intravenously, and/or subretinally.

In some embodiments, the composition can be administered at any dose suitable to treat at least one symptom in the equine (e.g., blindness or vision loss). For example, the composition can be administered at a dose of at least 0.1×10⁹ vg. In some embodiments, the composition is administered at a dose of at least 0.5×10⁹ vg. In some embodiments, the composition is administered at a dose of at least 1.0×10⁹ vg. In some embodiments, the composition is administered at a dose of at least 1.5×10⁹ vg. In some embodiments, the composition is administered at a dose of at least 2.0×10⁹ vg. In some embodiments, the composition is administered at a dose of at least 2.5×10⁹ vg. In some embodiments, the composition is administered at a dose of at least 3.0×10⁹ vg. In some embodiments, the composition is administered at a dose of at least 3.5×10⁹ vg. In some embodiments, the composition is administered at a dose of at least 4.0×10⁹ vg. In some embodiments, the composition is administered at a dose of at least 4.5×10⁹ vg. In some embodiments, the composition is administered at a dose of at least 5.0×10⁹ vg. In some embodiments, the composition is administered at a dose of at least 6.0×10⁹ vg. In some embodiments, the composition is administered at a dose of at least 7.0×10⁹ vg. In some embodiments, the composition is administered at a dose of at least 8.0×10⁹ vg. In some embodiments, the composition is administered at a dose of at least 9.0×10⁹ vg.

In some embodiments, the composition is administered in a single dose, and wherein the single dose treats and/or prevents at least one symptom associated with the ocular disease. In other embodiments, the composition is administered as part of a multi-dose regimen (i.e. more than one dose), and the dosing regimen treats and/or prevents at least one symptom associated with the ocular disease. In some embodiments, a multi-dose regimen includes administering a first dose, followed by at least a second dose. In some embodiments, for example, the second dose is administered about one month after the first dose, about three months after the first dose, about six months after the first dose, about one year after the first dose, about two years after the first dose, about three years after the first dose, about four years after the first dose, about five years after the first dose, about six years after the first dose, about seven years after the first dose, about eight years after the first dose, about nine years after the first dose, about ten years after the first dose. In some embodiments, the second dose is administered more than ten years after the first dose. In accordance with these embodiments, the multi-dose regimen can include a third, fourth, fifth, sixth, seventh, eighth, ninth, or tenth dose.

The AAV-IL-10 compositions of the present disclosure can include other components that may enhance the therapeutic efficacy of the composition. In some embodiments, the additional components can include excipients and/or adjuvants that enhance the delivery of the AAV-IL-10, including but not limited to, a surfactant. In some embodiments, the surfactant is a non-ionic surfactant, such as polysorbate 20, polysorbate 80, or polysorbate 85. Other surfactants can also be used, and at various concentrations, as would be recognized by one of ordinary skill in the art based on the present disclosure. As used herein, the term “surfactant” generally refers to organic substances having amphipathic structures (they are composed of groups of opposing solubility tendencies), typically an oil-soluble hydrocarbon chain and a water-soluble ionic group. Surfactants can be classified, depending on the charge of the surface-active moiety, into anionic, cationic and dispersing agents for various pharmaceutical compositions and preparations of biological materials. Suitable surfactants for use with the invention include, but are not limited to, non-ionic surfactants, ionic surfactants and zwitterionic surfactants. Typical surfactants for use with the invention include, but are not limited to, sorbitan fatty acid esters (e.g. sorbitan monocaprylate, sorbitan monolaurate, sorbitan monopalmitate), sorbitan trioleate, glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide): Cio-Cig alkyl sulfates (e.g. sodium cetyl sulfate, sodium lauryl sulfate, sodium oleyl sulfate), polyoxyethylene Cio-Cig alkyl ether sulfate with an average of 2 to 4 moles of ethylene oxide units added (e.g. sodium polyoxyethylene lauryl sulfate), and Ci-Ci₈ alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-18 fatty acids. A composition may include one or more of these surfactants. Preferred surfactants are polyoxyethylene sorbitan fatty acid esters e.g. polysorbate 20, 40, 60 or 80. Polysorbate 80 is particularly preferred.

Generally, the AAV-IL-10 compositions of the present disclosure contain a buffering agent (e.g., balanced salt solution) that is suitable for ocular administration. Suitable buffering agents for use with the invention include, but are not limited to, organic acid salts such as salts of citric acid, ascorbic acid, gluconic acid, carbonic acid, tartaric acid, succinic acid, acetic acid or phthalic acid; Tris, thomethamine hydrochloride, or phosphate buffer. In addition, amino acid components can also be used as buffering agent(s). For example, citrate or histidine buffers can be used. Additionally, the AAV-IL-10 compositions can include such buffering agent(s) or pH adjusting agent(s) to provide improved pH control. In some embodiments, the AAV-IL-10 compositions of the present disclosure can have a pH from about 4.0 to about 8.0, a pH from about 4.0 to about 7.0, a pH from about 4.0 to about 6.0, a pH from about 4.0 to about 5.0, a pH from about 5.0 to about 8.0, a pH from about 6.0 to about 8.0, a pH from about 7.0 to about 8.0, or a pH from about 5.0 to about 7.0.

The AAV-IL-10 compositions of the present disclosure can also comprise a biologically active agent, which can enhance the efficacy of the IL-10, and/or provide an additional therapeutic benefit. For example, in some embodiments, the biologically active agent is one or more of an immunosuppressant, an NSAID, a steroid, an antibacterial, and any combination thereof. In some embodiments, the steroid is dexamethasone or prednisone, and any combination thereof. In some embodiments, the NSAID is selected from the group consisting of flunixin meglumine, phenylbutazone, firocoxib, diclofenac, flurbiprofen, bromfenac, nepafenac, and any, combination thereof. In some embodiments, the immunosuppressant is selected from the group consisting of cyclosporin, tacrolimus (FK506), rapamycin (sirolomus), infliximab, bevacizumab, and any combination thereof. In some embodiments, the antibiotic is selected from the group consisting of gentamicin, tobramycin, amikacin, ceftazidime, vancomycin, and any combination thereof.

In some embodiments, the AAV-IL-10 compositions of the present disclosure can further comprise a polynucleotide encoding an immunomodulating agent. The immunomodulating polynucleotide can be integrated into the AAV vector, or it can be part of a separate gene delivery platform, including a separate AAV vector. In some embodiments, the immunomodulating polynucleotide encodes one or more of TGFβ, an IL-1 receptor antagonist, IL-33, IL-35, IL-37, IDO-1, and any combination thereof. Other immunomodulating polynucleotides can also be included, as would be recognized by one of ordinary skill in the art based on the present disclosure.

Embodiments of the present disclosure also include a kit comprising any of the compositions described herein, and at least one container. In some embodiments, the kit includes a device that can be used to administer any of the compositions described herein, including but not limited to, a syringe, an applicator, a depressor, and the like, to the eye of a subject. In some embodiments, the at least one container comprises a syringe and/or a needle suitable for administration to an eye of an equine. In some embodiments, the kit further comprises instructions for administration to an eye of an equine. In some embodiments, the instructions include steps for administering the compositions to an equine, including such information as dosing regimens, frequency of administration, routes of administration, side effects, and the like. In some embodiments, the kit includes a pre-filled syringe containing the AAV-IL-10 compositions. Generally, the syringe is filled with a therapeutically effective dose of the AAV-IL-10 composition that is sufficient to modulate one or more symptoms or conditions associated with the disease. The therapeutically effective dose does not have to completely cure the disease or completely eliminate symptoms. In some embodiments, the therapeutically effective dose can at least partially arrest the disease and its complications in a patient already suffering from the disease. Amounts effective for this use will depend upon the severity of the disorder being treated and the general state of the patient's own immune system.

### 3. Therapeutic Methods

Embodiments of the present disclosure also include a method of treating or preventing an ocular disease in equines using the equine IL-10 compositions described herein. In accordance with these embodiments, the method includes administering any of the compositions described herein to an equine. In some embodiments, the equine IL-10 polynucleotides of the present disclosure are administered by any means that are suitable to treat an ocular disease or condition. For example, equine IL-10 polynucleotides can be administered by injection of the composition into a portion of the subject's eye, and/or by direct application of the composition to a portion of the subject's eye. In some embodiments, the composition can be administered intravitreally (IVT), intracorneally, subconjunctivally, periocularly, suprachoroidally, intrasclerally, intravenously, and/or subretinally. In some embodiments, the composition can be administered at any dose suitable to treat at least one symptom in the equine (e.g., blindness or vision loss). For example, the composition can be administered at a dose of at least 0.1×10⁹ vg. In some embodiments, the composition is administered at a dose of at least 0.5×10⁹ vg. In some embodiments, the composition is administered at a dose of at least 1.0×10⁹ vg. In some embodiments, the composition is administered at a dose of at least 1.5×10⁹ vg. In some embodiments, the composition is administered at a dose of at least 2.0×10⁹ vg. In some embodiments, the composition is administered at a dose of at least 2.5×10⁹ vg. In some embodiments, the composition is administered at a dose of at least 3.0×10⁹ vg. In some embodiments, the composition is administered at a dose of at least 3.5×10⁹ vg. In some embodiments, the composition is administered at a dose of at least 4.0×10⁹ vg. In some embodiments, the composition is administered at a dose of at least 4.5×10⁹ vg. In some embodiments, the composition is administered at a dose of at least 5.0×10⁹ vg. In some embodiments, the composition is administered at a dose of at least 6.0×10⁹ vg. In some embodiments, the composition is administered at a dose of at least 7.0×10⁹ vg. In some embodiments, the composition is administered at a dose of at least 8.0×10⁹ vg. In some embodiments, the composition is administered at a dose of at least 9.0×10⁹ vg.

In some embodiments, the composition is administered in a single dose, and wherein the single dose treats and/or prevents at least one symptom associated with the ocular disease. In other embodiments, the composition is administered as part of a multi-dose regimen (i.e. more than one dose), and the dosing regimen treats and/or prevents at least one symptom associated with the ocular disease. In some embodiments, a multi-dose regimen includes administering a first dose, followed by at least a second dose. In some embodiments, for example, the second dose is administered about one month after the first dose, about three months after the first dose, about six months after the first dose, about one year after the first dose, about two years after the first dose, about three years after the first dose, about four years after the first dose, about five years after the first dose, about six years after the first dose, about seven years after the first dose, about eight years after the first dose, about nine years after the first dose, about ten years after the first dose. In some embodiments, the second dose is administered more than ten years after the first dose. In accordance with these embodiments, the multi-dose regimen can include a third, fourth, fifth, sixth, seventh, eighth, ninth, or tenth dose.

In some embodiments, the equine IL-10 compositions of the present disclosure are used to treat and/or prevent an ocular disease that is associated with blindness, impaired vision, and/or ocular pain. In accordance with these embodiments, administration of the composition treats and/or prevents the blindness, impaired vision, and/or ocular pain. In some embodiments, the treating and/or the preventing of blindness comprises lymphocyte suppression. In some embodiments, the ocular disease comprises uveitis, immune-mediated keratitis, heterochromic iridocyclitis with keratitis, endothelitis, posterior uveitis, chorioretinitis, optic neuritis, and any combination thereof. In some embodiments, the uveitis is recurrent, chronic, non-infectious uveitis.

### 4. Materials and Methods

Animals. Animals were handled in accordance with North Carolina University Institutional Animal Care and Use Committee (IACUC) and performed according to the Association for Research in Vision and Ophthalmology Statement for the Use of Animals in Ophthalmic and Visual Research. Lewis rats (female, n=14) (Charles River Labs, Wilmington, MA) were housed under 12/12 hour light/dark cycle in the NC State University Laboratory Animal Resources facility. Rats were randomly divided into 3 groups: the first and second groups had both eyes injected intravitreally with AAV8-equine-IL-10 at either a low dose 1.2×10⁹ viral genomes (vg) (n=5 rats), or a high dose, 1.2×10¹ vg (n=5 rats) and the third group of rats were injected with balanced salt solution (BSS, Alcon Labs) (n=4 rats) (see description of injections below). Experimental autoimmune uveitis (EAU) was induced in all rats 7 days following IVT injections. Serum for neutralizing antibody analysis was obtained from the lateral tail vein before intraocular injection and then obtained via intracardiac blood draw immediately after euthanasia. Serum collected was stored in −80° C. Daily blinded biomicroscopy examinations and every-other-day optical coherence tomography (OCT) assessed ocular abnormalities induced by IVT injections and following induction of EAU. Animals were sacrificed on day 21 and tissues were harvested for further analyses, as described below.

Intravitreal administration. For IVT injections, rats were anesthetized with 2-3% Isoflurane (Henry Schein) in oxygen to effect. Topical anesthetic, proparacaine HCL 0.1% (Bausch and Lomb), was applied topically to each eye prior to IVT injection. Animals were placed in lateral recumbency (left eye injected first followed by the right eye). Each eye was cleaned with dilute 5% betadine solution. Intraocular injections were performed under an operating microscope using a Hamilton syringe (Hamilton Co.) and a 34-gauge stainless steel needle. Three microliters of either viral suspension (low dose or high dose) or BSS, mixed with 0.01% fluorescein sodium salt (Sigma) was administered IVT in both eyes, with the needle placement 1 mm posterior to the temporal limbus. After injections were completed, intravitreal injection was verified (presence of fluorescence in the vitreous) followed by application of an antibiotic solution (Moxifloxacin 0.5%; Apotex Corp.) and ocular lubrication to the ocular surface to prevent infection and desiccation. Rats were recovered from anesthesia on a heating pad until fully ambulatory.

EAU Induction and clinical evaluation of EAU. Seven days after intravitreal injection, rats were immunized subcutaneously at the base of the tail (100 μg) and both thighs (50 μg) with a 1:1 volume of human interphotoreceptor retinoid binding protein, IRBP (AnaSpec Inc., Fremont, CA) and Complete Freunds Adjuvant, CFA (Sigma-Aldrich, St. Louis, MO).

Clinical assessment of ocular inflammation by slit lamp biomicroscopy (SL-17, KOWA) was performed daily with the examiner blinded to the treatment groups. Each eye was graded, according to previously described EAU scoring system: 0, normal; 0.5, dilated blood vessels in the iris; 1, abnormal pupil contraction; 2, hazy anterior chamber; 3, moderately opaque anterior chamber, but pupil still visible; 4, opaque anterior chamber and obscured pupil.

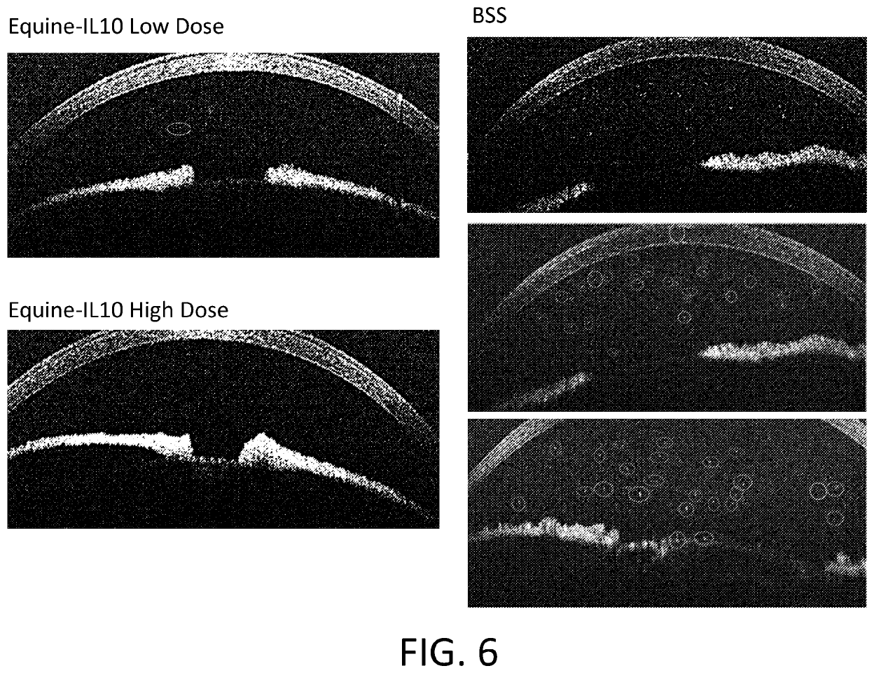

Optical coherence tomography of the ocular anterior segment. Spectral domain optical coherence tomography (SD-OCT) was performed to image the anterior segment of the eye (Envisu R-class SD-OCT; Bioptigen, Inc, Morrisville, NC). The SD-OCT contains a super-luminescent light emitting diode delivering a wavelength of 840 nm. Imaging was performed using the handheld probe of the SD-OCT device fitted with a noncontact 12-mm telecentric lens for image acquisition. After adjusting the arm reference length on the SD-OCT device by manufacturer recommendations, SD-OCT was set to 1000 A scans per B scan, and 100 B scans in total for each eye of each rat, to generate a radial volume of 4 mm in diameter. Each animal was manually restrained in right or left recumbency. Following imaging, cells in the anterior chamber were manually counted on 3 representative b-scan images of each eye, as previously described. Cell candidates within the anterior chamber were defined as at least two adjacent pixels with an intensity greater than a prespecified background threshold (FIG. 6).

Tissue collection. Immediately after euthanasia, the left eye of each rat was dissected and tissues collected. A strict tissue collection and cleaning procedures were used between sample collections to minimize the potential of cross-contamination. Control rats (BSS injected) were dissected first followed by rats treated with viral vector. Different sets of instruments were used to collect tissues for different treatment groups of rats. Instruments were cleaned with 70% alcohol, 5% sodium dodecyl sulfate detergent, and sterile saline between each sample taken. Upon collection, tissue samples were frozen on dry ice and then stored at −80° C. Specific ocular sections collected included the cornea, conjunctival, iris/ciliary body, retina, and optic nerve.

Quantification of transgene expression by qRT-PCR. DNA/RNA from the conjunctiva, cornea, retina, optic nerve, lens, and ciliary body/iris were extracted with the AllPrep DNA/RNA Mini Kit according to the kit protocol (Qiagen, Valencia. CA). Vector biodistribution was quantitatively analyzed by qPCR utilizing the Taqman probe technology. In short, the amount of vector-specific SV40 genome copies was standardized against an amplicon from a single copy rat gene, lamin B2, amplified from genomic DNA. For the detection of vector genomes, the plasmid (AAV-) standard curve was generated by serial ten-fold dilutions in 10 Mm Tris-HCl. qPCR was carried out with an initial denaturation step at 95° C. for 10 min, followed by 45 cycles of denaturation at 95° C. for 10 s, and annealing/extension at 60° C. for 45 s using SV40 polyA primers and an internal fluorescent probe (5′-fam AGCATITITTTCAC TGCATTCTAGTTGTGGTITGTC tamra-3′) (SEQ ID NO: 7). Genomic qPCR of rat lamin B2 was performed with LightCycler® 480 SYBR Green I Master with the following primers: forward primer 5′-GGACCCAAGGACTACCTCAAGGG-3′ (SEQ ID NO: 8); reverse primer 5′-AGGGCACCTCCATCTCGGAAAC-3′ (SEQ ID NO: 9). Purified and quantified mouse genomic DNA was used as a standard. The qPCR was carried out with an initial denaturation step at 95° C. for 10 min, followed by 45 cycles of denaturation at 95° C. for 10 s, and annealing first 5 cycles at 64° C. for 10 seconds then followed by a touch down PCR with a decrease of 2° C. every cycle for 10 s until it reaches the annealing at 60° C. for 10 seconds in the rest of the cycles. Extension was performed at 72° C. for 10 seconds. A melting curve analysis was performed at the end to ensure that a single product was amplified. Vector biodistribution data are reported as the number of double-stranded vector DNA (SV40) copies per μg of gDNA.

Histopathological Evaluation of EAU and Scoring. Animals were euthanized during peak disease activity (day 14 after induction of EAU), and the right eye was enucleated from each rat for histopathology. The globe from each rat was fixed in 4% buffered paraformaldehyde overnight at 4° C. and then transferred to 70% ethanol before embedding in paraffin. Eyes were sectioned 5 μm through the optic nerve horizontal plane, and stained with hematoxylin and eosin. Blinded infiltrative and structural grades of each eye were scored as previously described. The globes were scored by two blinded observers separately and averaged to determine infiltrative and structural scores for each rat. Data are presented as mean+/−standard deviation (SD).

AAV neutralizing antibody assay. To determine if intravitreal injection of AAV vectors results in an antibody response to the injected capsid, a neutralizing antibody assays were performed on HEK 293 cells using a previously reported method. In short, cells were seeded in 48-well plate at 25,000 cell/well in duplicate on the day before performing vector transduction. The next day, the pre- and post-injection serum was used 1:1 and then serially diluted 1:2 to 1:256 in DPBS to a final volume of 13 ul and incubated with AAV8 CMV-Firefly Luciferase titer to 8×10⁸ total viral genomes per replicate in 13 ul DPBS for 2 hours at 4° C. Serum/vector mixture was then added to cells and a luciferase assay was performed 48 hours post transduction using Promega Luciferase Assay System (Bright-Glo; Promega. Madison, WI) using a Perkin Elmer Victor 3 1420 Multi-label Counter Luminometer. Results were plotted to find the point at which the serum dilution suppressed transduction to less than 50% of pre-injection serum levels.

Statistical analysis. Comparisons of clinical and histologic scores from the experiments described herein were analyzed initially using the non-parametric Kruskal-Wallis test, i.e., to determine if at least one sample dominated. If there was a significant difference, then pairwise Wilcoxon (Mann-Whitney) tests were performed to evaluate for group differences using JMP version 14.0 (SAS Institute Cary, NC). qPCR data generated from the experiments of the present disclosure were analyzed with GraphPad Prism, version 8.0, using the Mann-Whitney test. Significance was set at p<0.05 for all comparisons.

### 5. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

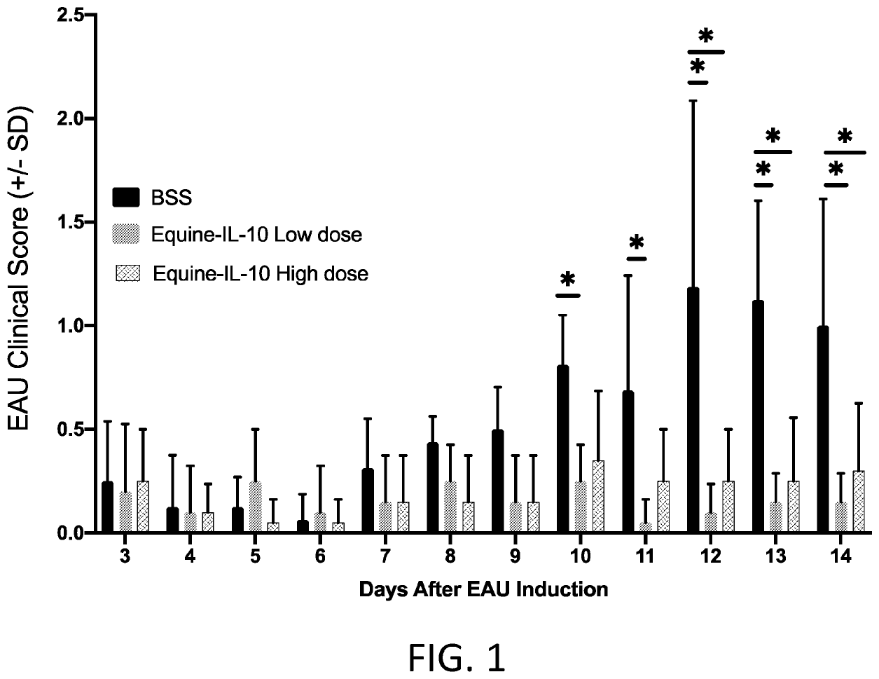

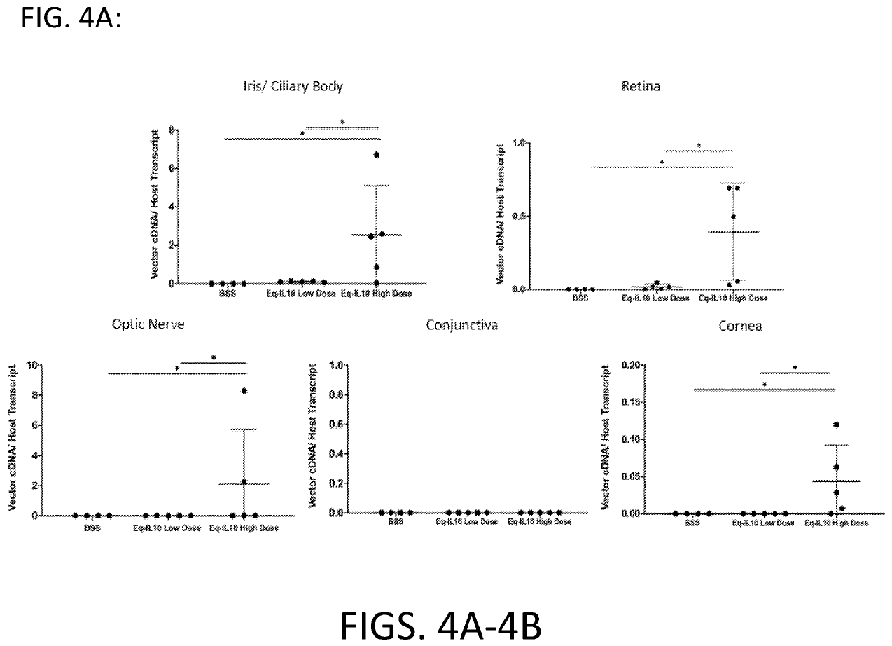

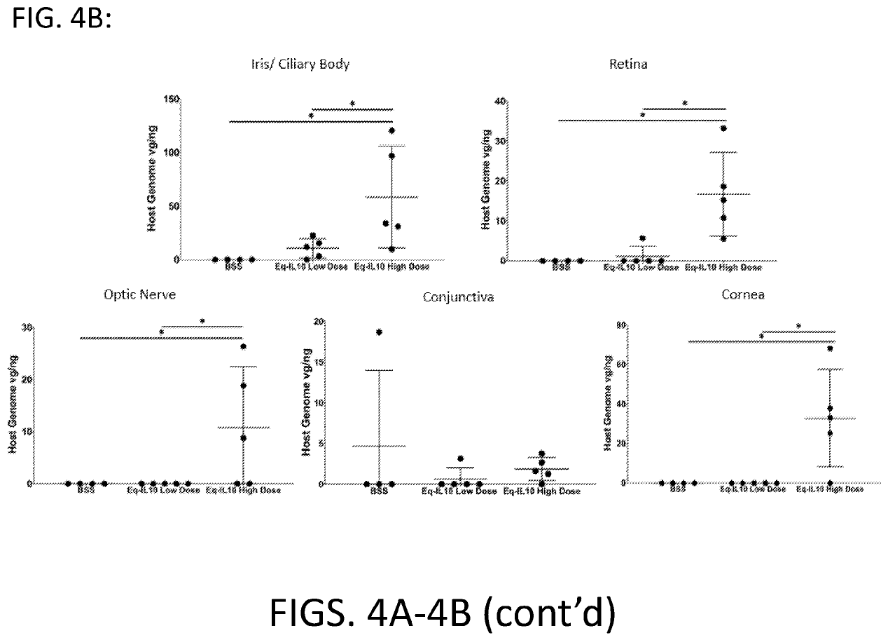

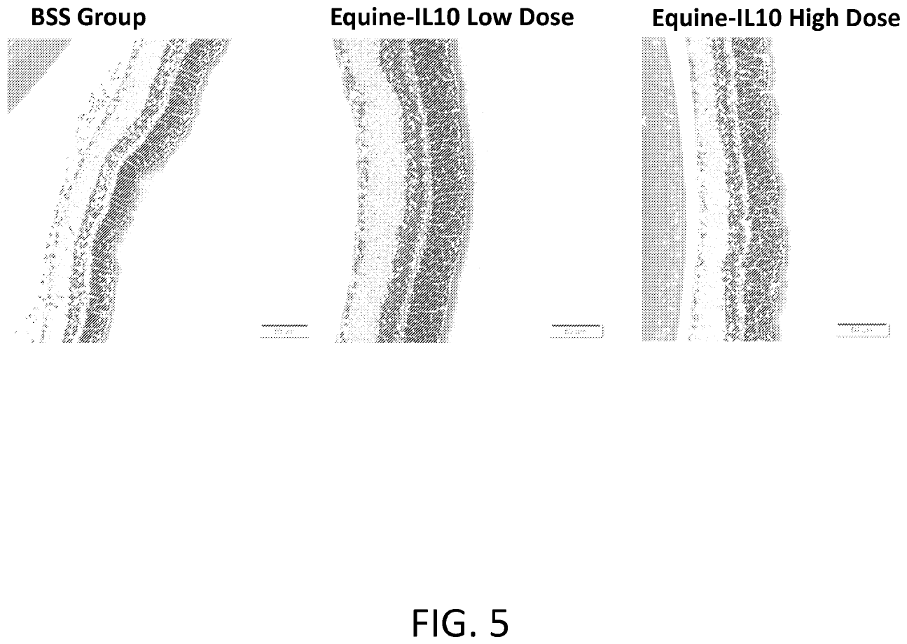

AAV-Equine-IL-10 gene therapy reduces inflammation in EAU rats. Rats were injected intravitreally in both eyes one week prior to induction of EAU. Clinical scores following IVT and prior to subcutaneous IRBP injection revealed very mild ocular inflammation (50.5 clinical inflammatory score) (see FIG. 4). Following EAU induction, ocular inflammation in BSS-dosed rats developed starting on day 9 after IRBP injection and peaked on days 12 and 13 (FIG. 1). Clinical EAU scores revealed that intravitreal AAV8-Equine-IL-10 consistently resulted in attenuated ocular inflammation as compared to the BSS dosed EAU rats (FIG. 1). Mean EAU clinical scores were significantly lower on days 12-14 in the AAV Equine-IL-10 high dose (1.2×10¹⁰ vg) treated rats compared to the BSS rats. And mean EAU clinical scores were significantly lower on days 10-14 in the AAV-Equine-IL-10 low dose (1.2×10⁹ vg) treated rats compared to BSS treated rats. (p=0.002 to 0.049); Pairwise Wilcoxon tests). There were no significant differences in mean EAU scores between eyes dosed with AAV Equine-IL-10 high dose or low dose on any experimental day.

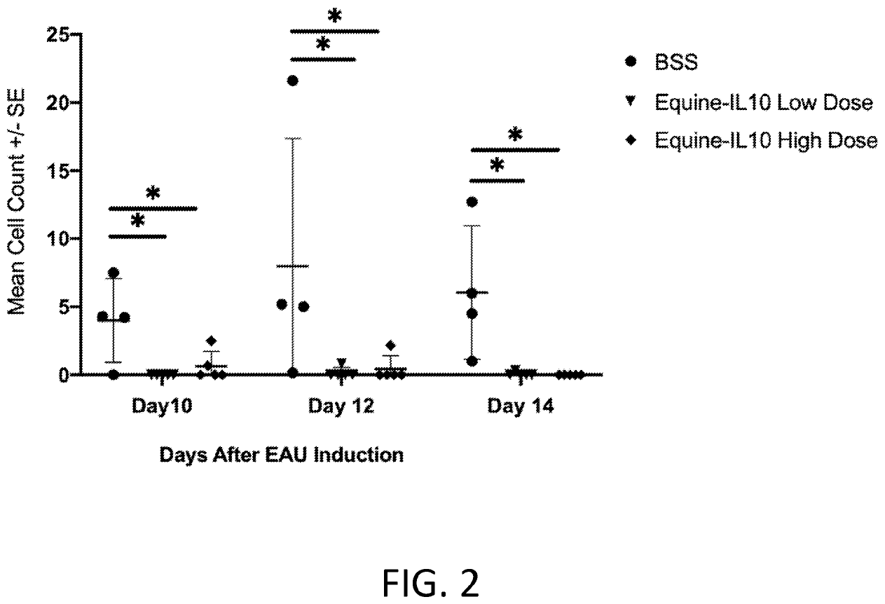

Optical coherence tomography (OCT) was performed prior IVT injections, once prior induction of EAU and then every other day following induction of EAU (FIG. 2). No AH cells were noted in any rat eye prior to IVT injection or prior to EAU induction. Mean inflammatory OCT AH cell counts were significantly less in rats treated with IVT AAV8-Equine-IL-10 (high or low dose) compared to BSS rats on days 10, 12 and 14 post EAU induction (p=<0.004 to 0.043); (Oneway ANOVA). There were no significant differences in mean inflammatory cell counts between AAV8-Equine-IL-10 high or low dose treated rats on any experimental day.

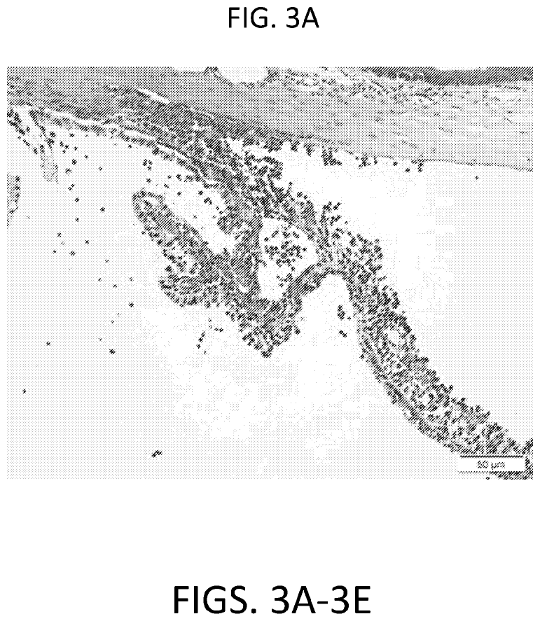

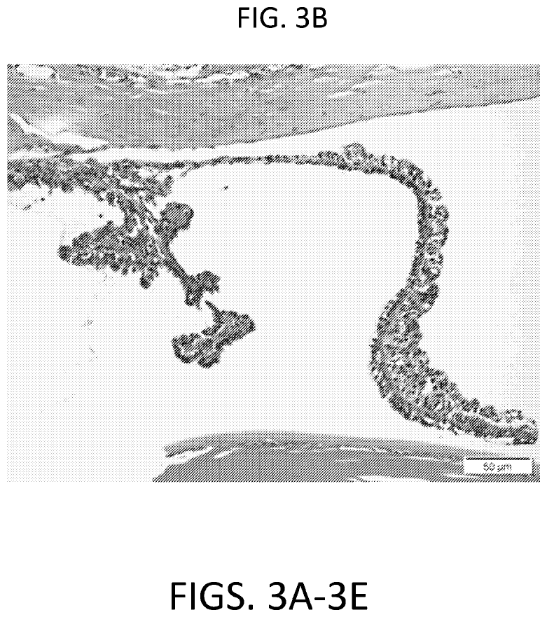

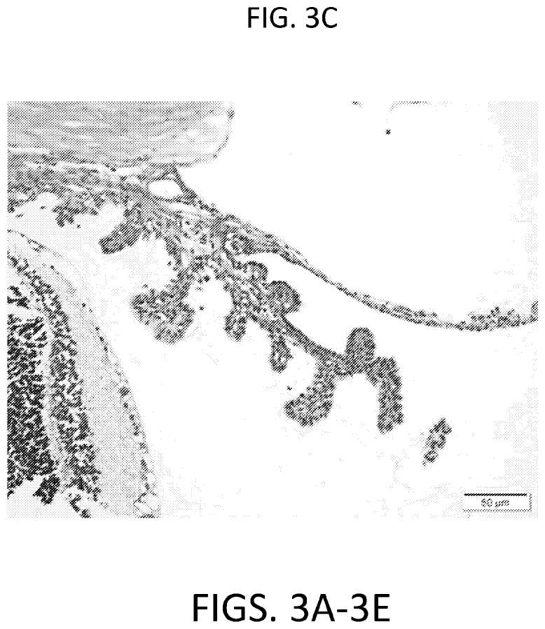

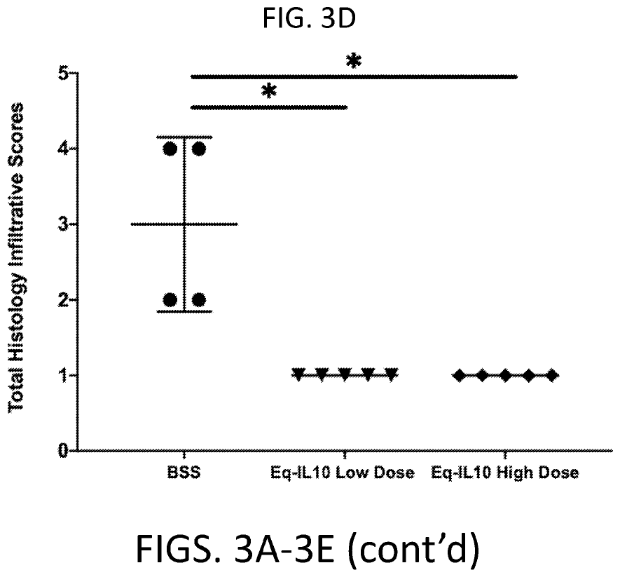

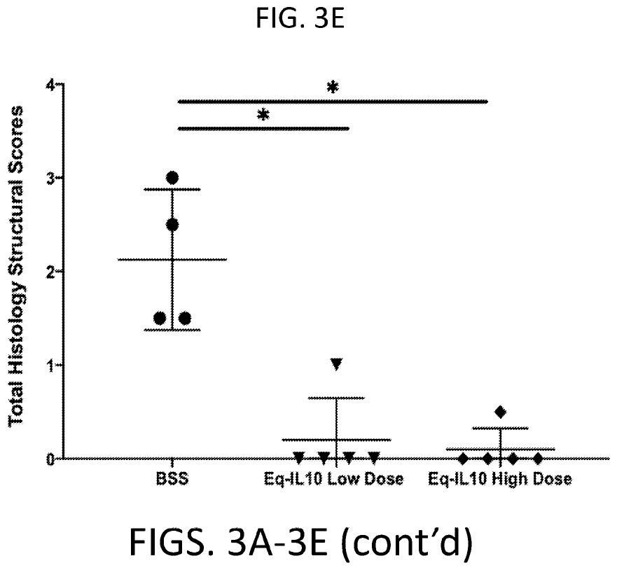

Following examination and scoring on day 14 after EAU induction, the right eye of each rat was fixed, sectioned, stained with H&E, and graded in a blinded manner by two experienced examiners using a well-established scoring system, which includes infiltrative and structure assessment scores. Histological examination in BSS dosed EAU eyes demonstrated severe intraocular inflammation as evidenced by iris thickening, and severe inflammatory cell infiltration in the ciliary body, iris, aqueous humor and vitreous, as well as moderate vasculitis formation. However, in both the high and low dose AAV8-Equine-IL-10 eyes, only a few scattered inflammatory cells were observed (FIGS. 3A-3C). The mean infiltrative and structural histological scores were significantly less in the AAV8-Equine-IL-10 low dose treated eyes treated eyes (mean±SD=1.0±0.0, 0.2±0.45 [infiltrative and structural, respectively]), as well as AAV8-Equine-IL-10 high dose treated eyes (1.0±0.0, 0.4±0.45) compared to the non-treated BSS-EAU eyes (3.5±, 3.0±0.82) (Wilcoxon; p=0.01, p=0.015) (FIGS. 3C-3D).

### Example 2

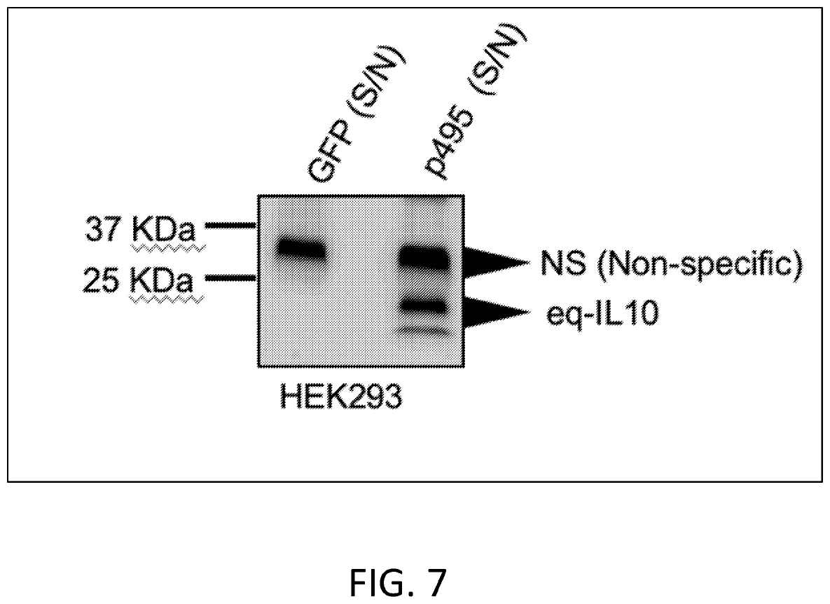

Equine IL-10 lymphocyte suppression. FIG. 7 includes representative images of IL-10 Western blots demonstrating expression of IL-10 in the supernatants of HEK293 cells. Plasmids were transfected similarly to previous ELISA experiment (20 ug of total protein was loaded in each lane). The primary antibody (R&D #AF1605) was used at 1:1000 dilution in 3% BSA, and incubated overnight at 4 C. The secondary antibody (R&D #HAF017) was used at 1:5000 dilution in 3% BSA and incubated for 1 hr at RT. Images were developed using Super Signal Femto (Fisher #34094).

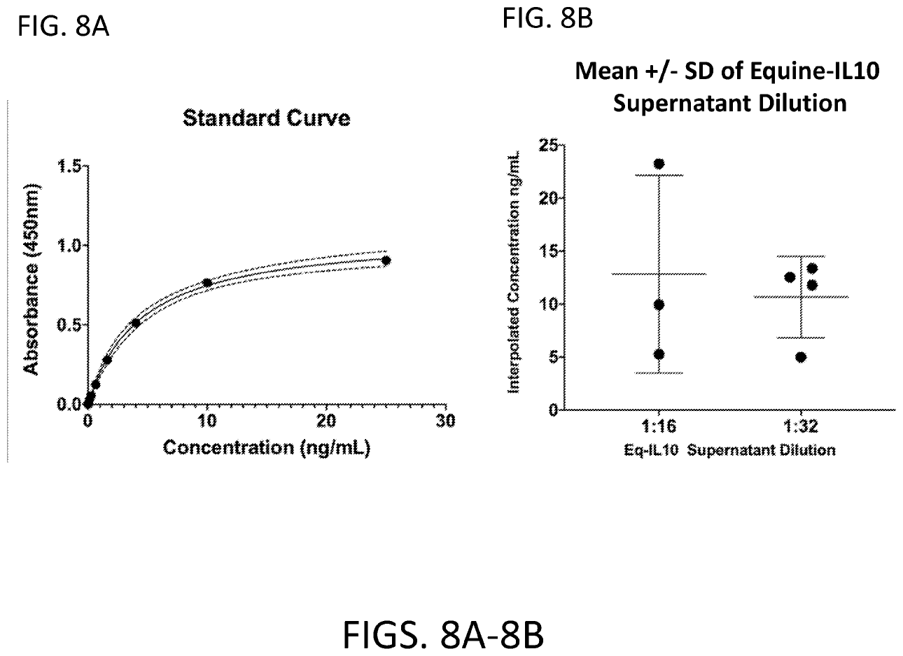

Next, experiments were conducted to determine the concentration of IL-10 in the supernatants of HEK293 cells using ELISA assays. FIGS. 8A-8B includes representative data from equine IL-10 ELISA assays. Interpolated data from equine-IL-10 dilution 1:16 had a higher standard deviation and had data points higher than the standard curve; therefore, data from the 1:32 dilution was used to determine the concentration of the Equine-IL-10 supernatant (A). Average Interpolated data from Equine-IL-10, 1:32 dilution=9.7 ng/mL concentration. Final supernatant concentration of Equine-IL-10 (interpolated concentration multiplied by dilution factor: 32) was approximately 310.4 ng/mL (B).

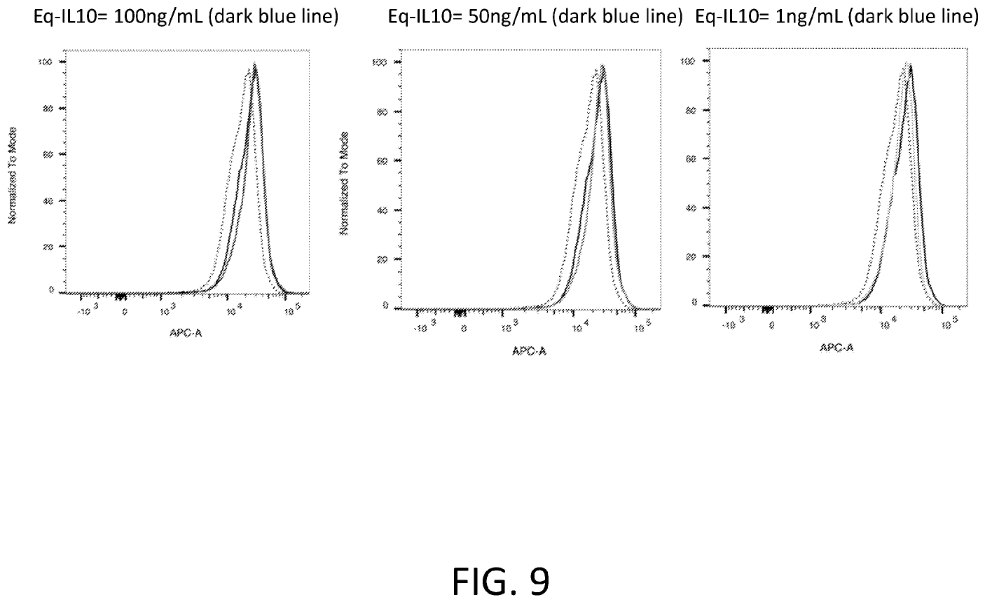

Additionally, experiments were conducted to test the effects of IL-10 on T-lymphocytes isolated from equine plasma. FIG. 9 includes representative data demonstrating equine IL-10 suppression of T-lymphocytes (using the supernatants described above). T-lymphocytes were extracted from equine plasma and incubated for 4 days in at ˜37° C. with 3 different concentrations of Equine-IL-10 supernatant (100 ng/mL, 50 ng/mL and 1 ng/mL). Controls included T-lymphocytes+ConA w/o equine-IL-10 (positive control); and T-lymphocytes+HEK cell supernatant w/o Equine-IL-10. As shown in FIG. 9, these results demonstrate that all three concentrations suppressed T-lymphocytes more than the positive control (dotted line). The 100 ng/mL concentration of Equine IL-10 suppressed proliferation more than the control supernatant (solid black line). The 50 ng/mL and 1 ng/mL concentrations did not suppress the T-Lymphocytes more than the control supernatant.

### Example 3

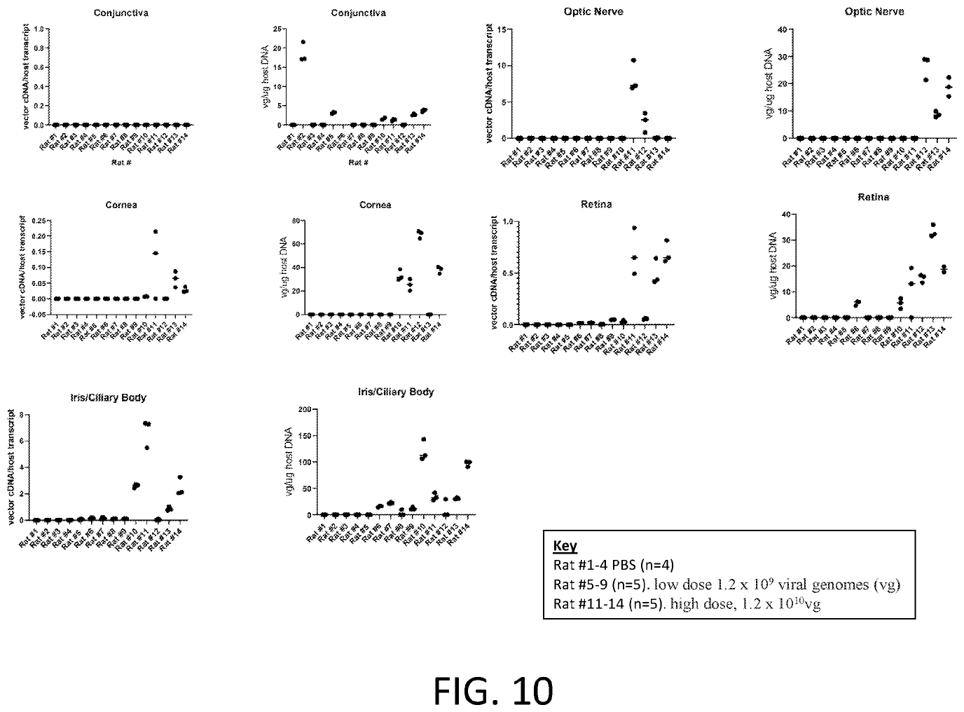

Equine-IL-10 ocular expression following intravitreal injection. Expression studies were also performed in various ocular tissues in the rat EAU model. Rats were administered AAV8-eqIL-10 by intravitreal injection at a low (1.2×10⁹ vg) or a high (1.2×10¹⁰ vg) dose (FIG. 10). Equine-IL-10 expression in treated rats was confirmed by RT-qPCR using RNA recovered from the iris/ciliary body and retina in the left eye of each rat (5 eyes per group) of both high and low dose treatment groups. In the low dose group, Equine-IL-10 cDNA transcript was detected in the iris/ciliary body 2/5 rats (40%), the conjunctiva in 1/5 rats (10%), and the retina in 1/5 rats (10%). In the high dose group, Equine-IL-10 transcript was detected in the iris/ciliary body 5/5 rats (100%), the cornea in 5/5 rats (100%), the optic nerve in 3/5 rats (60%), the conjunctiva in 4/5 rats (80%), and the retina in 4/5 rats (80%). In the high dose group, Equine-IL-10 cDNA expression was a detected in the cornea in 4/5 rats (80%), and in the optic nerve in 2/5 rats (40%).

Taken together, the vector bome eqIL-10 cDNA was significantly detected in the high dose cohort in the retina, cornea, iris/ciliary body, and optic nerve. This vector genome biodistribution analysis demonstrated dose dependent detection of eqIL-10 sequence in the retina and iris/ciliary body, while vector genomes were only detected in the cornea and optic nerve at the high dose.

### 6. Sequences

The various embodiments described herein make reference to the following nucleotide and amino acid sequences.

Equine IL-10 optimized nucleotide sequence (SEQ ID NO: 1):
ATGCACAGCTCCGCCCTGCTGTGCTACCTGGTGTTCCTGGCAGGAGTGG
GAGCAAGCCGGGACCGCGGCACCCAGAGCGAGAACTCCTGTACCCACTTCCCCA
CCAGCCTGCCTCACATGCTGCACGAGCTGAGGGCAGCCTTCTCCAGGGTGAAGA
CCTTCTTCCAGATGAAGGACCAGCTGGACAACATGCTGCTGAACGGCAGCCTGCT
GGAGGACTTCAAGGGATACCTGGGATGCCAGGCCCTGTCCGAGATGATCCAGTT
CTACCTGGAGGAAGTGATGCCACAGGCCGAGAACCACGGCCCCGACATCAAGGA
GCACGTGAACTCCCTGGGCGAGAAGCTGAAGACCCTGAGGGTGAGACTGAGGAG
ATGCCACAGGTTCCTGCCCTGTGAGAACAAGAGCAAGGCCGTGGAGCAGGTGAA
GAGCGCCTTCTCCAAGCTGCAGGAGAAGGGCGTGTACAAGGCCATGTCCGAGTT
CGACATCTTCATCAACTACATCGAGGCCTACATGACCACCAAGATGAAGAAC
Equine IL-10 optimized polypeptide sequence (SEQ ID NO: 2):
MHSSALLCYLVFLAGVGASRDRGTQSENSCTHFPTSLPHMLHELRAAFSR
VKTFFQMKDQLDNMLLNGSLLEDFKGYLGCQALSEMIQFYLEEVMPQAENHGPDIK
EHVNSLGEKLKTLRVRLRRCHRFLPCENKSKAVEQVKSAFSKLQEKGVYKAMSEFD
IFINYIEAYMTTKMKN
Equine IL-10 wt (SEQ ID NO: 3):
ATGCACAGCTCAGCACTGCTATGTTACCTGGTCTTCCTGGCCGGGGTGG
GAGCCAGCCGAGACCGGGGCACCCAGTCTGAGAACAGCTGCACCCACTTCCCAA
CCAGCCTGCCCCACATGCTCCATGAGCTCCGAGCCGCCTTCAGCAGGGTGAAGA
CTTTCTTTCAAATGAAGGACCAGCTGGACAACATGTTGTTGAACGGGTCCCTGCT
GGAGGACTTTAAGGGTTACCTGGGTTGCCAAGCCTTGTCGGAGATGATCCAGTTT
TACCTGGAGGAGGTGATGCCCCAGGCTGAGAACCACGGCCCAGACATCAAGGAG
CACGTGAACTCCCTGGGGGAAAAGCTGAAGACCCTCCGAGTGAGGCTGCGGCGC
TGTCATCGATTTCTGCCCTGTGAAAATAAGAGCAAGGCAGTGGAGCAGGTGAAG
AGTGCCTTCAGTAAGCTCCAAGAGAAAGGTGTCTACAAAGCCATGAGTGAGTTT
GACATCTTCATCAACTACATAGAAGCCTATATGACAACGAAGATGAAAAACTGA
AGCATCCTAGGGAACGAAGCATCCAGGACGGTGACTCTACTAGACTCTAGGACA
TAAATTGGAGATCTCCCAAATCCCATCCAGGGTTCTGGGAGAGCTGAATCAGCTC
CTTGGAAAACCCTGTGGTACCTCTCTCCTGAATATTTATTAACTCTGATACCTCAA
CTCCTATTTCTATTTATTTACTGAGCTTCTCTGTGAA
Mouse IL-10 (SEQ ID NO: 4):
ACATTTAGAGACTTGCTCTTGCACTACCAAAGCCACAAGGCAGCCTTGC
AGAAAAGAGAGCTCCATCATGCCTGGCTCAGCACTGCTATGCTGCCTGCTCTTAC
TGACTGGCATGAGGATCAGCAGGGGCCAGTACAGCCGGGAAGACAATAACTGCA
CCCACTTCCCAGTCGGCCAGAGCCACATGCTCCTAGAGCTGCGGACTGCCTTCAG
CCAGGTGAAGACTTTCTTTCAAACAAAGGACCAGCTGGACAACATACTGCTAAC
CGACTCCTTAATGCAGGACTTTAAGGGTTACTTGGGTTGCCAAGCCTTATCGGAA
ATGATCCAGTTTTACCTGGTAGAAGTGATGCCCCAGGCAGAGAAGCATGGCCCA
GAAATCAAGGAGCATTTGAATTCCCTGGGTGAGAAGCTGAAGACCCTCAGGATG
CGGCTGAGGCGCTGTCATCGATTTCTCCCCTGTGAAAATAAGAGCAAGGCAGTG
GAGCAGGTGAAGAGTGATTTTAATAAGCTCCAAGACCAAGGTGTCTACAAGGCC
ATGAATGAATTTGACATCTTCATCAACTGCATAGAAGCATACATGATGATCAAAA
TGAAAAGCTAAAACACCTGCAGTGTGTATTGAGTCTGCTGGACTCCAGGACCTAG
ACAGAGCTCTCTAAATCTGATCCAGGGATCTTAGCTAACGGAAACAACTCCTTGG
AAAACCTCGTTTGTACCTCTCTCCGAAATATTTATTACCTCTGATACCTCAGTTCC
CATTCTATTTATTCACTGAGCTTCTCTGTGAACTATTTAGAAAGAAGCCCAATATT
ATAATTTTACAGTATTTATTATTTTTAACCTGTGTTTAAGCTGTTTCCATTGGGGA
CACTTTATAGTATTTAAAGGGAGATTATATTATATGATGGGAGGGGTTCTTCCTT
GGGAAGCAATTGAAGCTTCTATTCTAAGGCTGGCCACACTTGAGAGCTGCAGGG
CCCTTTGCTATGGTGTCCTTTCAATTGCTCTCATCCCTGAGTTCAGAGCTCCTAAG
AGAGTTGTGAAGAAACTCATGGGTCTTGGGAAGAGAAACCAGGGAGATCCTTTG
ATGATCATTCCTGCAGCAGCTCAGAGGGTTCCCCTACTGTCATCCCCCAGCCGCT
TCATCCCTGAAAACTGTGGCCAGTTTGTTATTTATAACCACCTAAAATTAGTTCTA
ATAGAACTCATTTTTAACTAGAAGTAATGCAATTCCTCTGGGAATGGTGTATTGT
TTGTCTGCCTTTGTAGCAGACTCTAATTTTGAATAAATGGATCTTATTCG
Human IL-10 v1 (SEQ ID NO: 5):
ACACATCAGGGGCTTGCTCTTGCAAAACCAAACCACAAGACAGACTTG
CAAAAGAAGGCATGCACAGCTCAGCACTGCTCTGTTGCCTGGTCCTCCTGACTGG
GGTGAGGGCCAGCCCAGGCCAGGGCACCCAGTCTGAGAACAGCTGCACCCACTT
CCCAGGCAACCTGCCTAACATGCTTCGAGATCTCCGAGATGCCTTCAGCAGAGTG
AAGACTTTCTTTCAAATGAAGGATCAGCTGGACAACTTGTTGTTAAAGGAGTCCT
TGCTGGAGGACTTTAAGGGTTACCTGGGTTGCCAAGCCTTGTCTGAGATGATCCA
GTTTTACCTGGAGGAGGTGATGCCCCAAGCTGAGAACCAAGACCCAGACATCAA
GGCGCATGTGAACTCCCTGGGGGAGAACCTGAAGACCCTCAGGCTGAGGCTACG
GCGCTGTCATCGATTTCTTCCCTGTGAAAACAAGAGCAAGGCCGTGGAGCAGGT
GAAGAATGCCTTTAATAAGCTCCAAGAGAAAGGCATCTACAAAGCCATGAGTGA
GTTTGACATCTTCATCAACTACATAGAAGCCTACATGACAATGAAGATACGAAAC
TGAGACATCAGGGTGGCGACTCTATAGACTCTAGGACATAAATTAGAGGTCTCC
AAAATCGGATCTGGGGCTCTGGGATAGCTGACCCAGCCCCTTGAGAAACCTTATT
GTACCTCTCTTATAGAATATTTATTACCTCTGATACCTCAACCCCCATTTCTATTT
ATTTACTGAGCTTCTCTGTGAACGATTTAGAAAGAAGCCCAATATTATAATTTTTT
TCAATATTTATTATTTTCACCTGTTTTTAAGCTGTTTCCATAGGGTGACACACTAT
GGTATTTGAGTGTTTTAAGATAAATTATAAGTTACATAAGGGAGGAAAAAAAAT
GTTCTTTGGGGAGCCAACAGAAGCTTCCATTCCAAGCCTGACCACGCTTTCTAGC
TGTTGAGCTGTTTTCCCTGACCTCCCTCTAATTTATCTTGTCTCTGGGCTTGGGGC
TTCCTAACTGCTACAAATACTCTTAGGAAGAGAAACCAGGGAGCCCCTTTGATGA
TTAATTCACCTTCCAGTGTCTCGGAGGGATTCCCCTAACCTCATTCCCCAACCACT
TCATTCTTGAAAGCTGTGGCCAGCTTGTTATTTATAACAACCTAAATTTGGTTCTA
GGCCGGGCGCGGTGGCTCACGCCTGTAATCCCAGCACTTTGGGAGGCTGAGGCG
GGTGGATCACTTGAGGTCAGGAGTTCCTAACCAGCCTGGTCAACATGGTGAAAC
CCCGTCTCTACTAAAAATACAAAAATTAGCCGGGCATGGTGGCGCGCACCTGTA
ATCCCAGCTACTTGGGAGGCTGAGGCAAGAGAATTGCTTGAACCCAGGAGATGG
AAGTTGCAGTGAGCTGATATCATGCCCCTGTACTCCAGCCTGGGTGACAGAGCAA
GACTCTGTCTCAAAAAATAAAAATAAAAATAAATTTGGTTCTAATAGAACTCAGT
TTTAACTAGAATTTATTCAATTCCTCTGGGAATGTTACATTGTTTGTCTGTCTTCA
TAGCAGATTTTAATTTTGAATAAATAAATGTATCTTATTCACATCA
Human IL-10 v2 (SEQ ID NO: 6):
AGTCCCTTCGGGGAGGCTTCTGGTGAAGGAGGATCGCTAGAACCAAGC
TGTCCTCTTAAGCTAGTTGCAGCAGCCCCTCCTCCCAGCCACCTCCGCCAATCTCT
CACTCACCTITGGCTCCTGCCCTTAGGGTTACCTGGGTTGCCAAGCCTTGTCTGAG
ATGATCCAGTTTTACCTGGAGGAGGTGATGCCCCAAGCTGAGAACCAAGACCCA
GACATCAAGGCGCATGTGAACTCCCTGGGGGAGAACCTGAAGACCCTCAGGCTG
AGGCTACGGCGCTGTCATCGATTTCTTCCCTGTGAAAACAAGAGCAAGGCCGTGG
AGCAGGTGAAGAATGCCTTTAATAAGCTCCAAGAGAAAGGCATCTACAAAGCCA
TGAGTGAGTTTGACATCTTCATCAACTACATAGAAGCCTACATGACAATGAAGAT
ACGAAACTGAGACATCAGGGTGGCGACTCTATAGACTCTAGGACATAAATTAGA
GGTCTCCAAAATCGGATCTGGGGCTCTGGGATAGCTGACCCAGCCCCTTGAGAA
ACCTTATTGTACCTCTCTTATAGAATATTTATTACCTCTGATACCTCAACCCCCAT
TTCTATTTATTTACTGAGCTTCTCTGTGAACGATTTAGAAAGAAGCCCAATATTAT
AATTTTTTTCAATATTTATTATTTTCACCTGTTTTTAAGCTGTTTCCATAGGGTGAC
ACACTATGGTATTTGAGTGTTTTAAGATAAATTATAAGTTACATAAGGGAGGAAA
AAAAATGTTCTTTGGGGAGCCAACAGAAGCTTCCATTCCAAGCCTGACCACGCTT
TCTAGCTGTTGAGCTGTTTTCCCTGACCTCCCTCTAATTTATCTTGTCTCTGGGCTT
GGGGCTTCCTAACTGCTACAAATACTCTTAGGAAGAGAAACCAGGGAGCCCCTTT
GATGATTAATTCACCTTCCAGTGTCTCGGAGGGATTCCCCTAACCTCATTCCCCA
ACCACTTCATTCTTGAAAGCTGTGGCCAGCTTGTTATTTATAACAACCTAAATTTG
GTTCTAGGCCGGGCGCGGTGGCTCACGCCTGTAATCCCAGCACTTTGGGAGGCTG
AGGCGGGTGGATCACTTGAGGTCAGGAGTTCCTAACCAGCCTGGTCAACATGGT
GAAACCCCGTCTCTACTAAAAATACAAAAATTAGCCGGGCATGGTGGCGCGCAC
CTGTAATCCCAGCTACTTGGGAGGCTGAGGCAAGAGAATTGCTTGAACCCAGGA
GATGGAAGTTGCAGTGAGCTGATATCATGCCCCTGTACTCCAGCCTGGGTGACAG
AGCAAGACTCTGTCTCAAAAAATAAAAATAAAAATAAATTTGGTTCTAATAGAA
CTCAGTTTTAACTAGAATTTATTCAATTCCTCTGGGAATGTTACATTGTTTGTCTG
TCTTCATAGCAGATTTTAATTTTGAATAAATAAATGTATCTTATTCACATCA

Percent identity matrix produced using the various nucleotide sequences provided above:

| | Wthv1 | wthv2 | wtmouse | eq-opt | eq-wt |
|---|---|---|---|---|---|
| 1: wt-humanIL-10v1 | 100.00 | 93.97 | 69.48 | 79.03 | 86.11 |
| 2: wt-humanIL-10v2 | 93.97 | 100.00 | 64.54 | 66.74 | 73.92 |
| 3: wt-mouseIL-10 | 69.48 | 64.54 | 100.00 | 74.91 | 79.39 |
| 4: EquineIL10opt | 79.03 | 66.74 | 74.91 | 100.00 | 84.64 |
| 5: wt-horseIL-10 | 86.11 | 73.92 | 79.39 | 84.64 | 100.00 |

What is claimed is:

1. A composition for treating ocular disease in equines comprising:
an adeno-associated virus (AAV) vector comprising a polynucleotide encoding an equine IL-10 polypeptide, or a functional derivative or variant thereof; and
a pharmaceutically acceptable carrier and/or excipient;
wherein the composition is suitable for ocular administration to an equid.

2. The composition according to claim 1, wherein the polynucleotide encoding the equine IL-10 polypeptide is codon optimized.

3. The composition according to claim 1 or claim 2, wherein the polynucleotide encoding the equine IL-10 polypeptide is at least 75% identical to SEQ ID NO. 1.

4. The composition according to any of claims 1 to 3, wherein the polynucleotide encoding the equine IL-10 polypeptide comprises SEQ ID NO: 1.

5. The composition according to any of claims 1 to 4, wherein the IL-10 polypeptide has at least 85% identity with SEQ ID NO: 2.

6. The composition according to any of claims 1 to 5, wherein the IL-10 polypeptide comprises SEQ ID NO: 2.

7. The composition according to any of claims 1 to 6, wherein the AAV vector is at least one of an AAV serotype 1 (AAV1) vector, an AAV serotype 2 (AAV2) vector, an AAV serotype 3B (AAV3B) vector, an AAV serotype 4 (AAV4) vector, an AAV serotype 5 (AAV5) vector, an AAV serotype 6 (AAV6) vector, an AAV serotype 7 (AAV7) vector, an AAV serotype 8 (AAV8) vector, an AAV serotype 9 (AAV9) vector, or a derivative or variant thereof.

8. The composition according to any of claims 1 to 7, wherein the composition is administered by injection into a portion of the subject's eye or by direct application of the composition to a portion of the subject's eye.

9. The composition according to any of claims 1 to 8, wherein the composition is administered intravitreally (IVT), intracorneally, subconjunctivally, periocularly, suprachoroidally, intrasclerally, intracamerally, intravenously, and/or subretinally.

10. The composition according to any of claims 1 to 9, wherein the composition is administered at a dose of at least 1.0×10⁹ vg.

11. The composition according to any of claims 1 to 10 wherein the composition further comprises a buffer.

12. The composition according to any of claims 1 to 11, wherein the composition further comprises a surfactant.

13. The composition according to any of claims 1 to 12, wherein the composition comprises a pH from about 4.0 to about 8.0.

14. The composition of any of claims 1 to 13, wherein the composition further comprises a biologically active agent.

15. The composition of claim 14, wherein the biologically active agent is selected from the group consisting of an immunosuppressant, an NSAID, a steroid, an antibacterial, and any combination thereof.

16. The composition of claim 15, wherein the steroid is dexamethasone or prednisone, and any combination thereof.

17. The composition of claim 15, wherein the NSAID is selected from the group consisting of flunixin meglumine, phenylbutazone, firocoxib, diclofenac, flurbiprofen, bromfenac, nepafenac, and any combination thereof.

18. The composition of claim 15, wherein the immunosuppressant is selected from the group consisting of cyclosporin, tacrolimus (FK506), rapamycin (sirolimus), infliximab, bevacizumab, and any combination thereof.

19. The composition of claim 15, wherein the antibiotic is selected from the group consisting of gentamicin, tobramycin, amikacin, ceftazidime, vancomycin, and any combination thereof.

20. The composition of any of claims 1 to 19, wherein the AAV vector further comprises a polynucleotide encoding an immunomodulating agent selected from the group consisting of: TGFβ, an IL-1 receptor antagonist, IL-33, IL-35, IL-37, IDO-1, and any combination thereof.

21. A kit comprising any of the compositions of claims 1 to 20, and at least one container.

22. The kit of claim 21, wherein the at least one container comprises a syringe and a needle suitable for administration to an equine.

23. The kit according to claim 21 or claim 22, further comprising instructions for administration to an equine.

24. A method of treating or preventing an ocular disease in equines, the method comprising administering any of the compositions of claims 1 to 20 to an equine.

25. The method according to claim 24, wherein the ocular disease causes blindness, impaired vision, and/or ocular pain, and wherein the administration of the composition treats and/or prevents the blindness, impaired vision, and/or ocular pain.

26. The method according to claim 24, wherein the treating and/or the preventing of blindness comprises lymphocyte suppression.

27. The method according to any of claims 24 to 26, wherein the ocular disease comprises uveitis, immune-mediated keratitis, heterochromic iridocyclitis with keratitis, endothelitis posterior uveitis, chorioretinitis, optic neuritis, and any combination thereof.

28. The method according to claim 27, wherein the uveitis is recurrent, chronic, non-infectious uveitis.

29. The method according to any of claims 24 to 28, wherein the composition is administered by injection into a portion of the subject's eye or by direct application of the composition to a portion of the subject's eye.

30. The method according to any of claims 24 to 29, wherein the composition is administered intravitreally (IVT), intracorneally, subconjunctivally, periocularly, suprachoroidally, intrasclerally, intracamerally, intravenously, and/or subretinally.

31. The method according to any of claims 24 to 30, wherein the composition is administered at a dose of at least 1.0×10⁹ vg.

32. The method according to any of claims 24 to 31, wherein the composition is administered in a single dose, and wherein the single dose treats and/or prevents at least one symptom associated with the ocular disease.

33. The method of claim 32, wherein the at least one symptom comprises ocular cloudiness, blindness, impaired vision, and/or ocular pain.


SEQUENCE LISTING (9 sequences)

SEQ ID NO: 1 - DNA, 534 bp, Artificial sequence
  Synthetic
atgcacagctccgccctgctgtgctacctggtgttcctggcaggagtgggagcaagccgggaccgcggcacccagagcgagaactcctgtacccacttccccaccagcctgcctcacatgctgcacgagctgagggcagccttctccagggtgaagaccttcttccagatgaaggaccagctggacaacatgctgctgaacggcagcctgctggaggacttcaagggatacctgggatgccaggccctgtccgagatgatccagttctacctggaggaagtgatgccacaggccgagaaccacggccccgacatcaaggagcacgtgaactccctgggcgagaagctgaagaccctgagggtgagactgaggagatgccacaggttcctgccctgtgagaacaagagcaaggccgtggagcaggtgaagagcgccttctccaagctgcaggagaagggcgtgtacaaggccatgtccgagttcgacatcttcatcaactacatcgaggcctacatgaccaccaagatgaagaac

SEQ ID NO: 2 - protein, 178 aa, Artificial sequence
  Synthetic
MHSSALLCYLVFLAGVGASRDRGTQSENSCTHFPTSLPHMLHELRAAFSRVKTFFQMKDQLDNMLLNGSLLEDFKGYLGCQALSEMIQFYLEEVMPQAENHGPDIKEHVNSLGEKLKTLRVRLRRCHRFLPCENKSKAVEQVKSAFSKLQEKGVYKAMSEFDIFINYIEAYMTTKMKN

SEQ ID NO: 3 - DNA, 739 bp, Equus caballus
atgcacagctcagcactgctatgttacctggtcttcctggccggggtgggagccagccgagaccggggcacccagtctgagaacagctgcacccacttcccaaccagcctgccccacatgctccatgagctccgagccgccttcagcagggtgaagactttctttcaaatgaaggaccagctggacaacatgttgttgaacgggtccctgctggaggactttaagggttacctgggttgccaagccttgtcggagatgatccagttttacctggaggaggtgatgccccaggctgagaaccacggcccagacatcaaggagcacgtgaactccctgggggaaaagctgaagaccctccgagtgaggctgcggcgctgtcatcgatttctgccctgtgaaaataagagcaaggcagtggagcaggtgaagagtgccttcagtaagctccaagagaaaggtgtctacaaagccatgagtgagtttgacatcttcatcaactacatagaagcctatatgacaacgaagatgaaaaactgaagcatcctagggaacgaagcatccaggacggtgactctactagactctaggacataaattggagatctcccaaatcccatccagggttctgggagagctgaatcagctccttggaaaaccctgtggtacctctctcctgaatatttattaactctgatacctcaactcctatttctatttatttactgagcttctctgtgaa

SEQ ID NO: 4 - DNA, 1306 bp, Mus musculus
acatttagagacttgctcttgcactaccaaagccacaaggcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagactctaattttgaataaatggatcttattcg

SEQ ID NO: 5 - DNA, 1630 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 6 - DNA, 1476 bp, Homo sapiens
agtcccttcggggaggcttctggtgaaggaggatcgctagaaccaagctgtcctcttaagctagttgcagcagcccctcctcccagccacctccgccaatctctcactcacctttggctcctgcccttagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 7 - DNA, 36 bp, Artificial sequence
  Synthetic
agcatttttttcactgcattctagttgtggtttgtc

SEQ ID NO: 8 - DNA, 23 bp, Artificial sequence
  Synthetic
ggacccaaggactacctcaaggg

SEQ ID NO: 9 - DNA, 22 bp, Artificial sequence
  Synthetic
agggcacctccatctcggaaac